(19) United States
(12) Patent Application Publication
(10) Pub. No.: US 2014/0194498 A1
(43) Pub. Date: Jul. 10, 2014

(54) POLYISOPRENYLATED TETRACYCLIC XANTHONE WITH ANTICANCER ACTIVITY

(71) Applicant: Hong Kong Baptist University, Hong Kong (HK)

(72) Inventors: Hongxi XU, Hong Kong (HK); Hong ZHANG, Hong Kong (HK); Zhichao XI, Hong Kong (HK); Hongsheng TAN, Hong Kong (HK); Kaixian CHEN, Hong Kong (HK); Zhaoxiang BIAN, Hong Kong (HK); Dajian YANG, Hong Kong (HK); Shilin CHEN, Hong Kong (HK); Aiping LU, Hong Kong (HK); Albert Sun Chi CHAN, Hong Kong (HK)

(73) Assignee: Hong Kong Baptist University, Hong Kong (HK)

(21) Appl. No.: 14/149,819

(22) Filed: Jan. 8, 2014

Related U.S. Application Data
(60) Provisional application No. 61/750,954, filed on Jan. 10, 2013.

(51) Int. Cl. C07D 311/78

(52) U.S. Cl. CPC C07D 311/78
USPC 514/453; 549/384

(57) ABSTRACT

A novel polyisoprenylated tetracyclic xanthone, named garciesculentone B, isolated from Garcinia esculenta Y. H. Li is disclosed in the present invention. This compound is shown to have significant cytotoxicity against various cancer cells. In addition, garciesculentone B is shown to have antimigration effect on various cancer cells such as human esophageal carcinoma cell. The experimental data supports that this compound is useful for developing into an anticancer drug.

### CROSS-REFERENCE TO RELATED APPLICATIONS

This application claims the benefit of U.S. Provisional Patent Application Ser. No. 61/750,954 filed on Jan. 10, 2013, which is hereby incorporated by reference in its entirety.

### FIELD OF INVENTION

The present invention is in the field of pharmaceuticals and chemical industries. In particular, the present invention relates to a novel compound extracted from Garcinia esculenta Y. H. Li exhibiting cytotoxicity against human cancer cells. More particularly, the present invention relates to a novel polyisoprenylated tetracyclic xanthone, named garciesculentone B, being isolated from Garcinia esculenta Y. H. Li which has significant cytotoxicities against and/or antimigration effect on various cancer cells. The present invention also relates to the use of garciesculentone B as active compounds in anticancer drug.

### BACKGROUND OF INVENTION

Nature has played a dominant role in anticancer drug discovery and development, and over 70% of the anticancer drugs currently available may be classified as natural-product inspired. The genus Garcinia are a rich source of compounds with diverse chemical structures, including xanthones, polyprenylated benzophenones, bioflavonoids, etc., which have been reported in Ting Kan, W. L.; Yin, C.; Xu, H. X.; Xu, G.; To, K. K.; Cho, C. H.; Rudd, J. A.; Lin, G., Anti-tumor effects of novel compound, guttiferone K, on colon cancer by p21Waf1/Cip1-mediated G(0)/G(1) cell cycle arrest and apoptosis. Int J Cancer 2012; (b) Xu, G.; Kan, W. L.; Zhou, Y.; Song, J. Z.; Han, Q. B.; Qiao, C. F.; Cho, C. H.; Rudd, J. A.; Lin, G.; Xu, H. X., Cytotoxic acylphloroglucinol derivatives from the twigs of Garcinia cowa. J Nat Prod 2010, 73 (2), 104-8; (c) Prasad, S.; Ravindran, J.; Sung, B.; Pandey, M. K.; Aggarwal, B. B., Garcinol potentiates TRAIL-induced apoptosis through modulation of death receptors and antiapoptotic proteins. Mol Cancer Ther 2010, 9 (4), 856-68; (d) Wang, X.; Chen, Y.; Han, Q.-b.; Chan, C.-y.; Wang, H.; Liu, Z.; Cheng, C. H.-k.; Yew, D. T.; Lin, M. C. M.; He, M.-l.; Xu, H.-x.; Sung, J. J. Y.; Kung, H.-f., Proteomic identification of molecular targets of gambogic acid: Role of stathmin in hepatocellular carcinoma. PROTEOMICS 2009, 9 (2), 242-253, to possess a wide range of biological activities, especially antitumor effects. Therefore, our research group has been focusing on the isolation of new natural agents with antitumor activities from more Garcinia species and the study on their action mechanism to find potential candidates for anticancer treatment.

Although in US patent application publication no. US 2010/0137421 caged xanthones from Garcinia plants was disclosed, the present application discloses a polyisoprenylated tetracyclic xanthone extracted from Garcinia esculenta Y. H. Li which is structurally and functionally different from the caged xanthones disclosed in '421 and other prior arts, and therefore it is novel and not known in the art.

Citation or identification of any reference in this section or any other section of this application shall not be construed as an admission that such reference is available as prior art for the present application.

### SUMMARY OF INVENTION

Accordingly, an objective of this invention is to provide a new polyisoprenylated tetracyclic xanthone, named garciesculentone B, which exhibits cytotoxicity and antimigration effect against various cancer cell lines and has potential to be developed as anticancer drugs. The cancer cell lines which the presently claimed compound exhibits cytotoxicity against and/or antimigration effect on include but not limited to human hepatocellular carcinoma, breast adenocarcinoma and esophageal carcinoma.

In accordance with one aspect of the present invention, there is provided garciesculentone B which exhibits cytotoxicity against various cancer cell lines and can be used as active compounds in drugs for cancer treatment.

In accordance with another aspect of the present invention, there is provided garciesculentone B which exhibits antimigration effect on various cancer cells without cytotoxicity and can be used as active compounds in drugs for cancer treatment.

A further aspect of the present invention relates to a method for extracting the garciesculentone B of the present invention from a natural plant. The natural plant belongs to Garcinia genus, and more specifically, is Garcinia esculenta Y. H. Li. The method includes extracting a plant extract from dried and powdered twigs of said natural plant by using an organic solvent, followed by drying the plant extract under vacuum to obtain a petroleum ether-soluble part while the remaining matters are refluxed with 80% EtOH which is further dried under vacuum to produce a residue which is suspended in water and extracted with EtOAc to obtain a EtOAc-soluble part, and isolating said compound from said petroleum ether-soluble part.

A yet further aspect of the present invention relates to a method for treating cancer including using a new polyisoprenylated tetracyclic xanthone, named garciesculentone B, which exhibits cytotoxicity and antimigration effect against various cancer cell lines and has potential to be developed as anticancer drugs. The cancer cell lines which the presently claimed compound exhibits cytotoxicity against and/or antimigration effect on include but not limited to human hepatocellular carcinoma, breast adenocarcinoma and esophageal carcinoma. The anticancer drugs may be administered orally, or through intravenous or intraperitoneal injection, to a subject in needs thereof.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the structure of garciesculentone B.

FIG. 2 shows key correlations observed in the HMBC (H→C) NMR spectra of garciesculentone B.

FIG. 3 shows wound healing assay of garciesculentone B on human esophageal carcinoma (ECA109) cells.

### DETAILED DESCRIPTION OF INVENTION

The present invention is not to be limited in scope by any of the specific embodiments described herein. The following embodiments are presented for exemplification only.

Garcinia is a genus native to southeastern Asia, southern Africa, and Polynesia, with a total of 21 species distributed in mainland China. This genus is a rich source of compounds with diverse chemical structures, including xanthones, polyprenylated benzophenones, bioflavonoids, etc., which have been reported to possess a wide range of biological activities, especially antitumor effects. Therefore, our research group focused on the isolation of new natural agents with antitumor activities from more Garcinia species and the study on their action mechanism to find potential candidates for anticancer treatment. Garcinia esculenta Y. H. Li is the endemic plant of People's Republic of China, which is mainly distributed in the western and north-western part of Yunnan province. To date, there are still no reports on phytochemical and pharmacological research of this species. In the present invention, the chemical constituents of the twigs of G. esculenta Y. H. Li are found to contain potential anticancer lead compounds. Bioassay-directed fractionation has been employed in the present invention to isolate a novel polyisoprenylated tetracyclic xanthone.

Compound Characterization

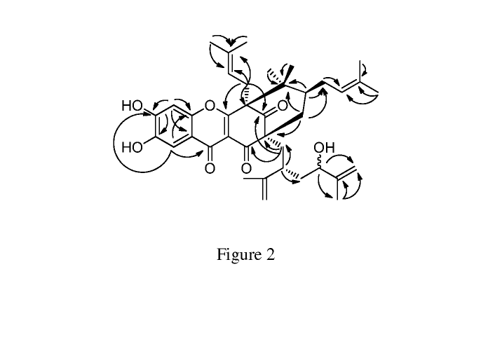

Garciesculentone B gives the molecular formula, C₃₈H₄₈O₇, as determined by HRESIMS. This elemental composition indicates 15 degrees of unsaturation, which shows that garciesculentone B has one more unit of unsaturation than garcimultiflorone. The ¹H NMR spectrum of garciesculentone B is comparable to that of garcimultiflorone, except for the loss of one aromatic proton. Two unique singlet aromatic proton signals at δ_{H} 6.91 and 7.43 are shown in the ¹H NMR aromatic region of garciesculentone B, which indicates the presence of a 1,2,4,5-tetrasubstituted benzene ring rather than a trisubstituted ring in garcimultiflorone. In addition, there are three oxygen-substituted aromatic carbons in the ¹³C NMR spectrum, which suggests that one aromatic proton of garcimultiflorone is substituted by oxygen in garciesculentone B. HMBC correlations of the proton signals at δ_{H} 6.91 and 7.43 to the carbon resonances at δ_{C} 147.8, 151.7, and 155.2, together with from the proton signal at δ_{H} 6.91 to the carbon resonance at δ_{C} 118.1, and from the proton signal at δ_{H} 7.43 to the carbon resonance at δ_{C} 173.4, allow to deduce that C-16 is oxygenated. Moreover, HMBC correlations between the carbon resonance at δ_{C} 194.7 and the proton resonances of H-8 (δ_{H} 2.03) and H-29 (δ_{H} 2.22), and between the carbon resonance at δ_{C} 176.8 and the proton resonances of H-17 (δ_{H} 2.90) allow the assignments of the signals at δ_{C} 194.7 and 176.8 to C-2 and C-4, respectively. The other key HMBC correlations are shown in the FIG. 2. Based on the information mentioned above, it is evident that the carbonyl at C-4 is enolized and that the oxygen is attached to C-16. Thus, the compound, garciesculentone B, represents the oxidized derivative of garcimultiflorone, and its planar structure is established.

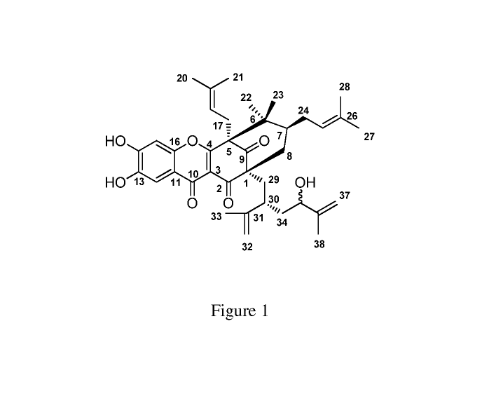

The relative configuration of garciesculentone B is determined by analysis of the 1D NMR data and the NOESY spectrum. The ¹³C NMR chemical shift of C-7 at δ_{C} 47.5 suggests that H-7 is α-oriented, or otherwise its signal would be located at δ_{C} 41.0-44.0. The C-22 methyl group and C-17 methylene group are determined to be both α-oriented from the correlations of CH₃-22/H-7 and CH₃-22/CH₂-17 found in the NOESY spectrum. Accordingly, the structure of garciesculentone B is established as shown in FIG. 1.

TABLE 1
¹³C and ¹H NMR Data (100 and 400 MHz, J in Hz)
of garciesculentone B^{a,b}

| position | δ_{C} | δ_{H,} mult. |
|---|---|---|
| 1 | 63.4 | |
| 2 | 194.7 | |
| 3 | 117.2 | |
| 4 | 176.8 | |
| 5 | 65.5 | |
| 6 | 50.9 | |
| 7 | 47.5 | 1.53, m |
| 8 | 45.1 | 2.20, m; 2.03, m |
| 9 | 209.2 | |
| 10 | 173.4 | |
| 11 | 118.1 | |
| 12 | 109.5 | 7.43, m |
| 13 | 147.8 | |
| 14 | 155.2 | |
| 15 | 103.9 | 6.91, m |
| 16 | 151.7 | |
| 17 | 27.0 | 2.9, m |
| 18 | 120.6 | 4.68, m |
| 19 | 136.2 | |
| 20 | 26.2 | 1.45, s |
| 21 | 18.8 | 1.77, s |
| 22 | 27.7 | 1.11, s |
| 23 | 23.8 | 1.27, s |
| 24 | 30.8 | 2.01, m; 1.79, m |
| 25 | 125.5 | 4.79, m |
| 26 | 133.9 | |
| 27 | 26.1 | 1.60, s |
| 28 | 18.1 | 1.34, s |
| 29 | 38.90^{c} | 2.22, m |
| 30 | 40.8 | 2.59, m |
| 31 | 149 | |
| 32 | 115.1 | 4.44, brs |
| 33 | 18.2 | 1.62, s |
| 34 | 38.98^{c} | 1.58, s; 1.47, m |
| 35 | 75.2 | 3.85, m |
| 36 | 147.8 | |
| 37 | 113.2 | 4.78, brs |
| 38 | 16.6 | 1.72, s |

^{a}Assignments are based on DEPT, HSQC, HMBC, and NOESY experiments; chemical shifts are given in ppm.
^{b}Measured in CD₃OD.
^{c}Data may be interchangeable.

TABLE 2
Cytotoxicity of garciesculentone B against Four Cancer Cell Lines^{a}

| Compound | SMMC-7721 | HepG 2 | MCF-7 | MDA-MB-231 |
|---|---|---|---|---|
| garciesculentone | 11.8 | 31.4 | 23.7 | 33.9 |
| B | | | | |
| Palcitaxel^{b} | 9.1 × 10⁻³ | 4.6 × 10⁻³ | 1.5 × 10⁻³ | 8.2 × 10⁻³ |

^{a}Results are expressed as IC₅₀ values in μM.
^{b}Positive control.

Bioassay

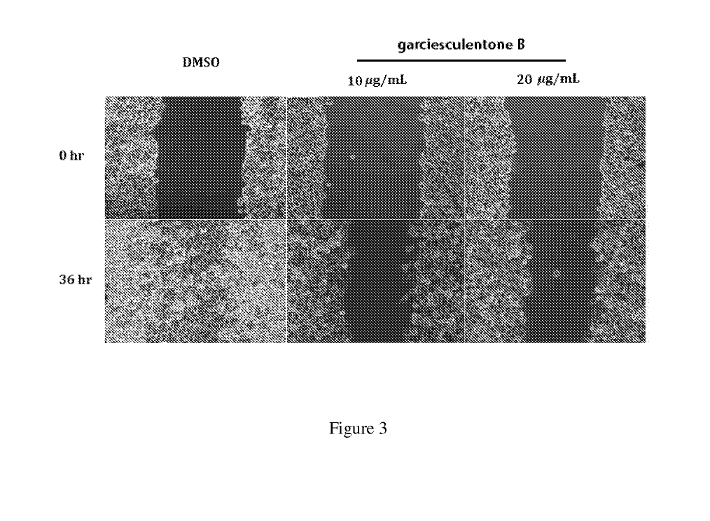

To determine the cytotoxicity of garciesculentone B, its IC₅₀ values on HepG2, SMMC-7721, MCF-7, and MDA-MB-231 cell lines are measured. As the results shown in Table 2, garciesculentone B has strong cell growth-inhibiting effect against SMMC-7721 cells with IC₅₀ value of 11.8 μM. In addition, further study is taken to test whether garciesculentone B can exert effects on cell motility. A wound healing assay using ECA109 cells is performed in this regard. Maintained in completed medium, a confluent monolayer of cells is scratched to form a wound and incubated in the absence or presence of garciesculentone B. After incubation, samples in the presence of 10 and 20 μg/mL garciesculentone B show a significant delay in cells moving into the wound area. In control group, the cells migrate into the wound area by 36 hours to such an extent that the wound edges are indistinguishable, whereas garciesculentone B treated cells do not migrate into and completely close the wound (FIG. 3).

### EXPERIMENTAL SECTION

General Experimental Procedures. Optical rotations were measured with a JASCO P-1020 polarimeter. Ultraviolet absorption spectra were recorded on a UV-2401 PC spectrophotometer. IR spectra were obtained from a Bio-Rad FtS-135 spectrometer. NMR spectra were measured on a Bruker AV-400 spectrometer with TMS as the internal standard. Mass spectrometry was performed on a Waters Q-TOF Premier instrument (Micromass MS Technologies, Manchester, UK) spectrometer, with an electrospray ion source (Waters, Milford, Mass.) connected to a lock-mass apparatus performing a real-time calibration correction. Column chromatography was performed with CHP20P MCI gel (75-150 μm, Mitsubishi Chemical Coparation, Japan), silica gel (200-300 mesh, Qingdao Haiyang Chemical Co., Ltd.), Sephadex LH-20 (GE Healthcare Bio-Sciences AB, Sweden), and reversed-phase C₁₈ silica gel (50 μm, YMC, Kyoto, Japan). Precoated TLC sheets of silica gel 60 GF254 (Qingdao Haiyang Chemical Co., Ltd.) were used. A waters 2535 Series machine equipped with a Xbridge C18 column (4.6×250 mm, 5 μm) was used for HPLC analysis, and preparative Xbridge Prep C₁₈ OBD column (19×250 mm, 5 μm) was used in sample preparation. Paclitaxel was purchased from Sigma-Aldrich Trading Co. Ltd. (Shanghai, People's Republic of China).

Plant Material. The twigs of Garcinia esculenta Y. H. Li were collected in Nujiang, Yunnan Province, People's Republic of China, in August 2010. The plant material was identified by Prof. Yuanchuan Zhou, Yunnan University of Traditional Chinese Medicine. A voucher specimen (Herbarium No. 20100801) has been deposited at the Innovative Research Laboratory of TCM, Shanghai University of Traditional Chinese Medicine.

Extraction and Isolation. Air-dried and powdered twigs of the plant (4 kg) were extracted with petroleum ether (5×20 L, each two days). The combined extracts were evaporated to dryness under vacuum to gain the petroleum ether-soluble part (fraction I, 40 g). The remaining materials were refluxed with 80% EtOH (v/v, 5×20 L). The combined extracts were evaporated to dryness under vacuum and the residue was suspended in H₂O (5 L) and extracted with EtOAc (5×5 L) to obtain fractions II (50 g, the EtOAc-soluble part) and III (the remaining H₂O part), respectively. The remaining materials were refluxed with distilled water (5×20 L) to gain the H₂O-soluble part (fraction IV). Fraction I was shown to possess the significant cytotoxic activities against four human cancer cell lines.

Fraction I (37 g) was chromatographed on a silica gel column chromatography (CC) using a gradient of petroleum ether—EtOAc (100:0 to 50:50, v/v), and yielded fifteen fractions (IA-IO) based on the TLC profiles. Fraction IN was chromatographed separately on MCI gel eluted with 90% and 100% EtOH, successively, to afford two subfractions (IN1 and IN2). Fraction IN1 was separated in a preliminary fashion using a reversed-phase C18 silica gel column eluted with MeOH—H₂O (70:30 to 100:0) as a gradient system to give five subfraction (IN1a-IN1e). Fraction IN1d were further purified by preparative HPLC (MeOH—H₂O, 85:15, with 0.1% formic acid in H₂O, 20 mL/min) to obtain garciesculentone B (4 mg).

Garciesculentone B: yellow gum; [α]²⁰_{D}+18.4 (c 0.04, MeOH); UV (MeOH) λₘₐₓ (logε) 264 (4.33), 334 (3.98) nm; IR (KBr) νₘₐₓ 3421, 2964, 2923, 2879, 1731, 1687, 1618, 1513, 1465, 1379, 1292, 1188, 1143, 895 cm⁻¹; ¹H NMR (CD₃OD, 400 MH_{Z}) data, see Table 1; ¹³C NMR (CD₃OD, 100 MH_{Z}) data, see Table 2; HRESIMS 615.3312 [M−H]⁻ (calcd for C₃₈H₄₇O₇, 615.3322).

Cytotoxicity Assay. Cytotoxic activitiy of garciesculentone B were evaluated by MTT assay using SMMC-7721 (human hepatocellular carcinoma), HepG2 (human hepatocellular carcinoma), MCF-7 (human breast adenocarcinoma), and MDA-MB-231 (human breast adenocarcinoma). Paclitaxel was used as positive control. The detailed methodology for cytotoxicity assay has already been described in Xia, Z.-X.; Zhang, D.-D.; Liang, S.; Lao, Y.-Z.; Zhang, H.; Tan, H.-S.; Chen, S.-L.; Wang, X.-H.; Xu, H.-X., Bioassay-Guided Isolation of Prenylated Xanthones and Polycyclic Acylphloroglucinols from the Leaves of Garcinia nujiangensis. Journal of Natural Products 2012.

Wound healing assay. A wound healing assay was performed by using a modified procedure described previously in Jones, J. I.; Gockerman, A.; Busby, W. H., Jr.; Wright, G.; Clemmons, D. R., Insulin-like growth factor binding protein 1 stimulates cell migration and binds to the alpha 5 beta 1 integrin by means of its Arg-Gly-Asp sequence. Proc Natl Acad Sci USA 1993, 90 (22), 10553-7. Briefly, cells were plated in 24 wells plate at 70% confluence in conditioned medium. 24 hours after seeding the monolayers were wounded by scoring with a sterile plastic 20 μL micropipette tip, then rinsed several times with media to remove cell debris and then incubated in completed medium in the absence or presence of tested compounds (10 and 20 μg/mL) for various periods of time up to 36 hours. Cell migration into the wound surface was monitored by Olympus IX71 microscopy and digitally photographed.

### INDUSTRIAL APPLICATION

The present invention discloses a novel extract of Garcinia esculenta Y. H. Li shown to have cytotoxicity against human cancer cells. In particular, the present invention relates to a novel polyisoprenylated tetracyclic xanthone, named garciesculentone B, isolated from the petroleum ether extract of the 80% (v/v) EtOH extract of Garcinia esculenta Y. H. Li. This new compound shows significant cytotoxicities against SMMC-7721 cells with IC₅₀ value of 11.8 μM. In addition, garciesculentone B shows antimigration effect on ECA109 cells by wound healing assay. The experimental results in the present invention support that this novel compound can be developed as anticancer drug.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A composition for treating cancer comprising a compound with a chemical structure of 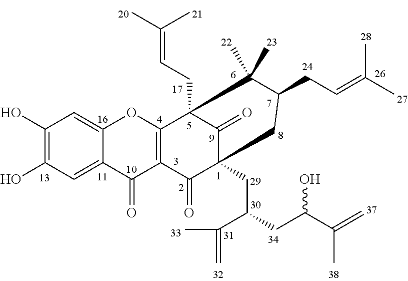
which is extracted from a natural plant.

2. The composition according to claim 1 wherein said compound comprises polyisoprenylated tetracyclic xanthone.

3. The composition according to claim 2 wherein said polyisoprenylated tetracyclic xanthone comprises garciesculentone B.

4. The composition according to claim 1 wherein the natural plant comprises a subspecies of the genus Garcinia.

5. The composition according to claim 4 wherein the subspecies of genus Garcinia is Garcinia esculenta Y. H. Li.

6. The composition according to claim 1 wherein said compound is extracted from the twigs of said plant.

7. The composition according to claim 1 wherein the cancer treated comprises human hepatocellular carcinoma, breast adenocarcinoma and esophageal carcinoma.

8. A method for preparing said composition of claim 1 comprising extracting a plant extract from dried and powdered twigs of said natural plant by using an organic solvent, followed by drying the plant extract under vacuum to obtain a petroleum ether-soluble part while the remaining matters are refluxed with 80% EtOH which is further dried under vacuum to produce a residue which is suspended in water and extracted with EtOAc to obtain a EtOAc-soluble part, and isolating said compound from said petroleum ether-soluble part.

9. A method for treating cancer comprising administering a composition containing a compound to a subject in needs thereof, said compound having a chemical structure of 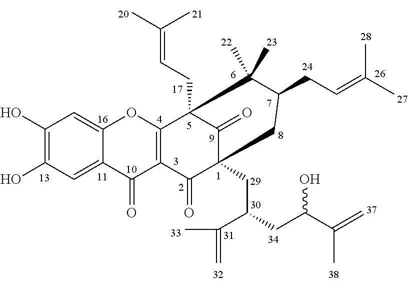
which is extracted from a natural plant.

10. The method according to claim 9 wherein said compound comprises polyisoprenylated tetracyclic xanthone.

11. The method according to claim 10 wherein said polyisoprenylated tetracyclic xanthone comprises garciesculentone B.

12. The method according to claim 9 wherein the natural plant comprises a subspecies of the genus Garcinia.

13. The method according to claim 12 wherein the subspecies of genus Garcinia is Garcinia esculenta Y. H. Li.

14. The method according to claim 9 wherein said compound is extracted from the twigs of said plant.

15. The method according to claim 9 wherein the cancer treated comprises human hepatocellular carcinoma, breast adenocarcinoma and esophageal carcinoma.

16. The method of claim 9 wherein said administering is via oral, intraperitoneal injection or intravenous injection.
